(19) United States
(12) Patent Application Publication
(10) Pub. No.: US 2010/0125107 A1
(43) Pub. Date: May 20, 2010

(54) MULTIPLE GASIFIERS MANIFOLDED TO MULTIPLE FISCHER-TROPSCH REACTORS WITH OPTIONAL RECYCLE TO THE REACTORS

(75) Inventors: Joshua G. LEE, Denver, CO (US); Mark IBSEN, Highland Ranch, CO (US); Robert F. HOGSETT, Arvada, CO (US); Harold A. WRIGHT, Longmont, CO (US)

Correspondence Address: CONLEY ROSE, P.C.;David A. Rose, P. O. BOX 3267, HOUSTON, TX, 77253-3267, US

(73) Assignee: RENTECH, INC., Los Angeles, CA (US)

(21) Appl. No.: 12/616,255

(22) Filed: Nov. 11, 2009

Related U.S. Application Data
(60) Provisional application No. 61/115,165, filed on Nov. 17, 2008.

(51) Int. Cl. C07C 27/00; C01B 3/00; B01J 8/04; B01J 10/00

(52) U.S. Cl. 518/702; 252/373; 422/188; 422/190

(57) ABSTRACT

A method of producing synthesis gas via gasification in a Fischer-Tropsch plant, the method including providing a number of gasifiers, the number of gasifiers provided being at least one more than the base number required to provide 100% plant capacity of synthesis gas when each gasifier is operated at 100% gasifier capacity. A method of continually producing synthesis gas via gasification of a carbonaceous feed in a Fischer-Tropsch plant by providing a number of gasifiers, the number of gasifiers provided being at least one more than the base number required to provide 100% plant capacity of synthesis gas when each gasifier is operated at 100% gasifier capacity; and adjusting the amount of synthesis gas produced by adjusting the number of online gasifiers, the flow rate of carbonaceous feed to each gasifier, or a combination thereof. A system for carrying out the method is also provided.

### CROSS-REFERENCE TO RELATED APPLICATIONS

This application claims the benefit under 35 U.S.C. §119(e) of U.S. Provisional Patent Application Ser. No. 61/115,165 filed Nov. 17, 2008, which is hereby incorporated herein by reference in its entirety.

### STATEMENT REGARDING FEDERALLY SPONSORED RESEARCH OR DEVELOPMENT

Not Applicable.

### BACKGROUND OF THE INVENTION

1. Technical Field

The present invention relates generally to Fischer-Tropsch production of hydrocarbons. More particularly, the present invention relates to a system and method for maintaining operation of a Fischer-Tropsch plant at near steady gas flow rates and full capacity; facilitating startup and turndown; and permitting continued operation of a Fischer-Tropsch plant at a desired capacity (e.g., full capacity) even during the event that a gasifier, an acid gas removal unit or AGR, a Fischer-Tropsch reactor, and/or a product upgrading or PU unit must be taken offline.

2. Background of the Invention

In Fischer-Tropsch processes, gasifiers are commonly utilized to produce synthesis gas from a carbonaceous feedsource. The low quality synthesis gas produced in the gasifiers is generally cleaned to produce high quality synthesis gas via units such as acid gas removal units, or AGRs. The high quality synthesis gas is introduced into Fischer-Tropsch reactors, in which catalytic synthesis of hydrocarbons from the synthesis gas occurs. Product upgrading units (PUs) are used to upgrade the Fischer-Tropsch hydrocarbons to desirable products.

Process units go on-stream and off-stream on a periodic basis. For example, gasifiers may be only about 80% reliable. It is desirable, however, to design Fischer-Tropsch plants that have on-stream efficiencies greater than this. Although a spare gasifier is typically on site should a gasifier go offline, spare gasifiers are typically not in operation.

In conventional configurations, there may be only 1 SRU (sulfur recovery unit), one AGR and two gasifiers. Consequently, special design considerations are imposed on the gasifier to be able to avoid reduced production while switching gasifiers. In such instances, it is necessary to be able to simultaneously shut one gasifier down while the other gasifier is brought online so the entire plant need not be shut down for any period of time.

Conventional designs comprising separate trains of gasifiers and Fischer-Tropsch reactors in parallel make maintenance of desired capacity during replacement/maintenance of a unit difficult, and complicate startup and turndown.

Accordingly, there is a need in industry for a system and method for maintaining 100% plant production in a Fischer-Tropsch process even in the event where a processing unit is taken offline. The system and method should also allow for simplified startup and turndown, allowing various units to be operated at different times and at different flow rates, as desired.

### SUMMARY

Herein disclosed is a method of producing synthesis gas via gasification in a Fischer-Tropsch plant, the method comprising providing a number of gasifiers, the number of gasifiers provided being at least one more than the base number required to provide 100% plant capacity of synthesis gas when each gasifier is operated at 100% gasifier capacity. The method may further comprise normally operating all of the number of gasifiers at a capacity whereby greater than 100% of the synthesis gas required by the plant is produced. Surplus synthesis gas may be stored in a tank. In applications, surplus synthesis gas is stored in a salt dome. The method may further comprise normally operating each of the number of gasifiers at less than 100% gasifier capacity while maintaining 100% Fischer-Tropsch plant capacity.

Also disclosed is a method of continually producing synthesis gas via gasification of a carbonaceous feed in a Fischer-Tropsch plant, the method comprising providing a number of gasifiers, the number of gasifiers provided being at least one more than the base number required to provide 100% plant capacity of synthesis gas when each gasifier is operated at 100% gasifier capacity, and adjusting the amount of synthesis gas produced by adjusting the number of online gasifiers, the flow rate of carbonaceous feed to each gasifier, or a combination thereof. Adjusting the amount of synthesis gas produced may further comprise taking at least one gasifier offline and increasing the amount of synthesis gas produced by the remaining online gasifiers such that 100% Fischer-Tropsch plant capacity is maintained.

A method of maintaining full production of product in a Fischer-Tropsch process is also disclosed, the method comprising: manifolding a plurality of Fischer-Tropsch reactors to a plurality of gasifiers in a Fischer-Tropsch process having a required synthesis gas production flow rate for full capacity, whereby the plurality of gasifiers minus one is capable of producing the required synthesis gas flow rate, and wherein the plurality of Fischer-Tropsch reactors minus one is capable of producing full capacity of product. The turndown capacity of each of the plurality of Fischer-Tropsch reactors may be such that a single Fischer-Tropsch reactor is capable of being operated with an amount of synthesis gas produced by one of the plurality of gasifiers during startup. The method may further comprise recycling at least a portion of a Fischer-Tropsch tail gas produced in the Fischer-Tropsch reactors to at least one of the Fischer-Tropsch reactors via at least one recycle compressor. In applications, the number of recycle compressors is less than the number of Fischer-Tropsch reactors. During startup, a single gasifier and a single Fischer-Tropsch reactor may be brought online prior to the remaining Fischer-Tropsch reactors and the remaining gasifiers being brought online. In applications, the method further comprises introducing the synthesis gas produced in the gasifiers to at least one acid gas removal unit upstream of the plurality of Fischer-Tropsch reactors, and introducing at least a portion of the Fischer-Tropsch product from the plurality of Fischer-Tropsch reactors into at least one product upgrading unit. During startup, a single gasifier, a single acid gas removal unit, a single Fischer-Tropsch reactor, and a single product upgrading unit may be brought online prior to the remaining units being brought online. Introducing the synthesis gas produced in the gasifiers to at least one acid gas removal unit may comprise introducing the synthesis gas produced in the gasifiers into a plurality of acid gas removal units, wherein at least one Fischer-Tropsch reactor, at least one gasifier, and at least one acid gas removal unit may be taken offline while maintaining full plant capacity. The method may further comprise introducing at least a portion of the Fischer-Tropsch product into a storage unit if the amount of Fischer-Tropsch product produced in the plurality of Fischer-Tropsch reactors is greater than the capacity of the online product upgrading units. In applications, the method comprises utilization of at least 6 Fischer-Tropsch reactors and at least 5 gasifiers. The method may further comprise utilization of at least two product upgrading units. The method may further comprise utilization of at least two acid gas removal units.

Also disclosed is a Fischer-Tropsch system comprising a plurality of gasifiers manifolded together, wherein the plurality of gasifiers have a synthesis gas production capacity greater than the required synthesis gas for operation of the Fischer-Tropsch plant at full capacity, whereby at least one of the plurality of gasifiers may be taken offline and the remaining reactors ramped up to maintain full plant production. The system may further comprise at least one of acid gas removal unit downstream of the plurality of gasifiers for the removal of sulfur. In applications, the system comprises a plurality of acid gas removal units. In embodiments, the Fischer-Tropsch system further comprises a plurality of Fischer-Tropsch reactors manifolded to the plurality of gasifiers, whereby at least one of the plurality of reactors may be taken offline and the remaining reactors ramped up to maintain full plant production. The system may further comprise a number of recycle compressors adapted to return Fischer-Tropsch tail gas to at least one of the Fischer-Tropsch reactors, wherein the number of recycle compressors is at least one less than the number of Fischer-Tropsch reactors. The system may comprise at least 4 Fischer-Tropsch reactors and at least two recycle compressors. In applications, the system further comprises at least one product upgrading unit downstream of the plurality of Fischer-Tropsch reactors. In embodiments, the system further comprises storage for Fischer-Tropsch product should at least one product upgrading unit be taken offline. The system may comprise a single product upgrading unit and storage for Fischer-Tropsch product should the single product upgrading unit be taken offline. The system may further comprise at least 2 acid gas removal units and comprising at least 5 gasifiers, at least 4 Fischer-Tropsch reactors, and at least one product upgrading unit.

These and other embodiments and potential advantages will be apparent in the following detailed description and drawings. Other uses of the disclosed system and method will become apparent upon reading the disclosure and viewing the accompanying drawings. While specific examples may be presented in the following description, other embodiments are also envisioned. The embodiments described herein are exemplary only, and are not intended to be limiting.

### BRIEF DESCRIPTION OF THE DRAWINGS

For a more detailed description of the preferred embodiment of the present invention, reference will now be made to the accompanying drawings, wherein:

FIG. 1 is a schematic of a Fischer-Tropsch system according to an embodiment of the invention.

FIG. 2 is a schematic of a Fischer-Tropsch system according to another embodiment of the invention.

FIG. 3a is a schematic of a conventional arrangement of gasifiers and operating capacities thereof.

FIG. 3b is a schematic of an arrangement of gasifiers according to an embodiment of this invention and a first selection of operating capacities thereof.

FIG. 3c is a schematic of an arrangement of gasifiers according to another embodiment of this invention and a second selection of operating capacities thereof.

FIG. 3d is a schematic of the arrangement of gasifiers according to the embodiment of FIG. 3b and a third selection of operating capacities thereof.

### NOTATION AND NOMENCLATURE

As used herein, the terms “syngas” and “synthesis gas” are used to refer to a gaseous stream comprising hydrogen and carbon monoxide. The “syngas” or “synthesis gas” stream may further comprise other components, for example, without limitation, the “syngas” or “synthesis gas” stream may comprise carbon dioxide, methane, etc.

Use of the term “spare” with reference to a unit of apparatus is meant to imply that the unit is an additional unit above and beyond the number of units required for production of a certain amount of product. For example, the phrase “spare gasifier” is used to refer to an additional gasifier above and beyond the number of gasifiers required for the production of a desired amount of synthesis gas. The use of the term “spare” is not meant to imply that the unit must be unutilized during normal operation. For example, a ‘spare’ gasifier may normally be online with the balance of the gasifiers, each gasifier working at full capacity or a fraction thereof.

### DETAILED DESCRIPTION

Overview. The invention is a system and process for the production of hydrocarbons from synthesis gas, allowing 100% plant production even in the event wherein one unit must be pulled offline. The system and process also permit simplified startup and turndown. In an embodiment, the invention is an improvement to a process for the production of synthesis gas, the improvement effected via the normal operation of an additional (or ‘spare’) gasifier allowing 100% plant production even in the event wherein one gasifier must be pulled offline.

System for Production of Fischer-Tropsch Hydrocarbons. The system comprises a plurality of gasifiers, with the number of gasifiers being at least one greater than the number of gasifiers required to produce the required plant synthesis gas when each gasifier is operating at 100% capacity. In embodiments, the system comprises a plurality of gasifiers manifolded to a plurality of Fischer-Tropsch reactors. Manifolding units together rather than running separate trains in parallel permits operation of the Fischer-Tropsch plant at 100% plant production even in the event that a processing unit must be taken offline. It also permits simplified startup and turndown, by allowing various units to be operated at different times and at different flow rates. The system may further comprise recycle to the multiple Fischer-Tropsch reactors and/or utilization of a spare gasifier.

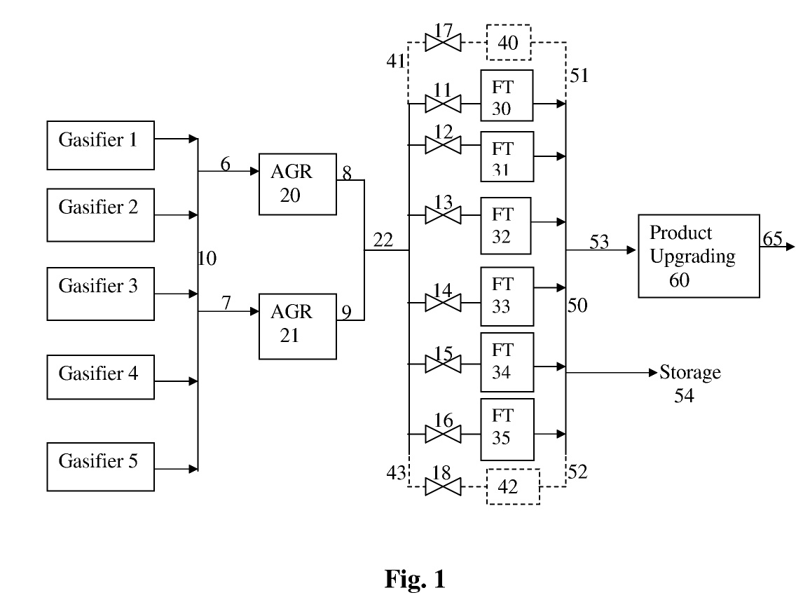

Description of the Fischer-Tropsch system will now be made with reference to FIG. 1 which is a schematic of a Fischer-Tropsch system 100. As mentioned above, the system comprises a plurality of gasifiers, with the number of gasifiers being at least one greater than the number of gasifiers required to produce the required plant synthesis gas when each gasifier is operating at 100% capacity. The system may comprise at least three gasifiers, at least four gasifiers, or at least five gasifiers. In the embodiment of FIG. 1, system 100 comprises five gasifiers, gasifiers 1, 2, 3, 4, and 5. Gasifiers 1-5 convert carbonaceous feedstock into synthesis gas. The carbonaceous feedstock may comprise coal, biomass, municipal solid waste (MSW), or a combination thereof.

Synthesis gas produced in gasifiers 1-5 is introduced into Fischer-Tropsch reactors for conversion to higher hydrocarbons. In certain applications, the Fischer-Tropsch system comprises at least two gasifiers and at least two Fischer-Tropsch reactors manifolded together. The gasifiers may be manifolded to at least three Fischer-Tropsch reactors, at least four Fischer-Tropsch reactors, at least five Fischer-Tropsch reactors, or at least six Fischer-Tropsch reactors. In embodiments, there may be 4-12 Fischer-Tropsch reactors. In embodiments, the plant comprises at least 10 Fischer-Tropsch reactors and the production is about 25,000 bpd. In embodiments, the plant comprises 4, 6, 8, or 10 Fischer-Tropsch reactors. In embodiments, the production is 10,000 bpd and the plant comprises 4 Fischer-Tropsch reactors. In the embodiment of FIG. 1, system 100 comprises six Fischer-Tropsch reactors, Fischer-Tropsch reactors 30, 31, 32, 33, 34, and 35.

The system may further comprise at least one acid gas removal unit. Furthermore, the Fischer-Tropsch system may further comprise at least one product upgrading unit. In the embodiment of FIG. 1, system 100 comprises two AGR units, 20 and 21, and one product upgrading unit 60. The system may further comprise compressors for the recycle of Fischer-Tropsch tail gas to the Fischer-Tropsch reactors. In embodiments, the number of compressors is less than the number of Fischer-Tropsch reactors.

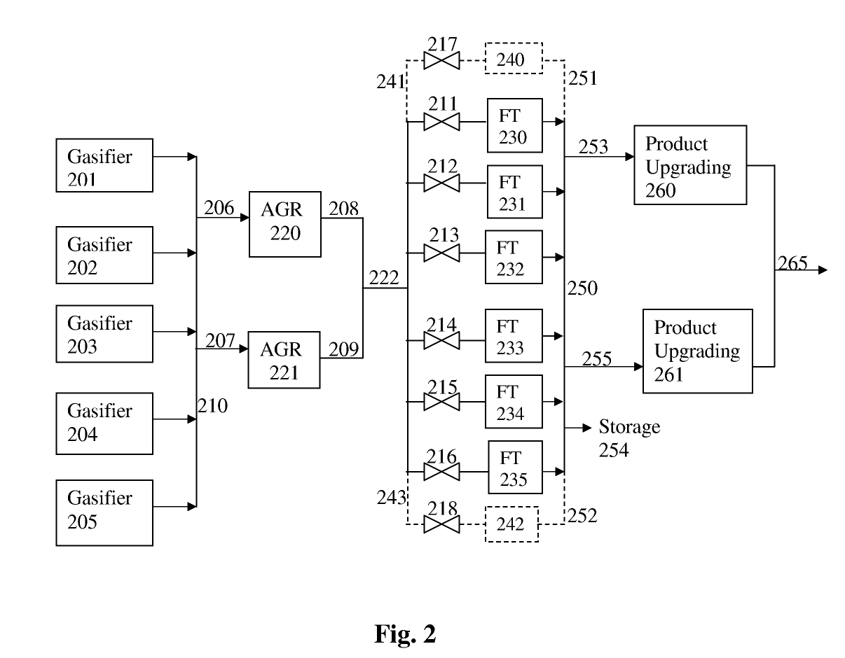

FIG. 2 is a schematic of a Fischer-Tropsch system according to another embodiment of this disclosure. In this embodiment, system 200 comprises two product upgrading units 260 and 261 in addition to five gasifiers, gasifiers 201, 202, 203, 204, and 205 manifolded to six Fischer-Tropsch reactors, Fischer-Tropsch reactors 230, 231, 232, 233, 234, and 235; and two acid gas removal units 220, and 221.

Gasifiers. System 100 comprises at least two gasifiers. The gasifiers may be any gasifier known in the art to be suitable for the conversion of carbonaceous feedstock into synthesis gas. The gasifier may comprise an inlet for carbonaceous feedstock. In embodiments, the feedstock comprises coal, MSW (municipal solid waste), biomass, E-fuel, RDF (refuse derived fuel), or a combination thereof. The gasifier may also comprise an inlet for steam, air, or oxygen. In embodiments, the gasifier is a counter-current fixed bed (up-draft) gasifier, a co-current fixed bed (down-draft) gasifier, a fluidized bed reactor, or an entrained flow gasifier.

Utilization of Spare Gasifier. In an embodiment, the invention is the utilization of a ‘spare’ gasifier in addition to the number of gasifiers needed to supply 100% of the required synthesis gas when operating at full capacity. In such an embodiment, the system may comprise a single Fischer-Tropsch reactor.

Fischer-Tropsch Reactors. System 100 may comprise at least two Fischer-Tropsch reactors. The Fischer-Tropsch reactor may be any suitable reactor known in the art for the conversion of synthesis gas into higher (C²⁺) hydrocarbons. In embodiments, the Fischer-Tropsch reactors are slurry reactors. As the Fischer-Tropsch reaction is highly exothermic, the Fischer-Tropsch reactor(s) may comprise internal or external heat exchangers to control the temperature of the reactor contents.

Fischer-Tropsch Catalyst. The reactor comprises Fischer-Tropsch catalyst effective for catalyzing the conversion of carbon monoxide and hydrogen into C²⁺ hydrocarbons. In embodiments, the catalyst comprises cobalt. In embodiments, the catalyst comprises iron. A suitable iron-based Fischer-Tropsch catalyst is disclosed in U.S. patent application Ser. No. 12/198,459, which is hereby incorporated herein to the extent that it provides details or explanations supplemental to those disclosed herein.

During Fischer-Tropsch conversion, the percent by weight of the disclosed iron catalyst in the Fischer-Tropsch reactor slurry (for example, in a slurry bubble column reactor, or SBCR) may be in the range of from 5 to 25 percent by weight of iron in the slurry, in the range of from 10 and 25 percent by weight, or about 20 percent by weight of the slurry.

Acid Gas Removal Units. System 100 may further comprise acid gas removal units. The AGRs may be any suitable apparatus known in the art for removing sulfur and/or sulfur-containing compounds from the sour synthesis gas produced in the gasifiers. In embodiments, AGR comprises a solvent system such as a Selexol system, a Rectisol system, or a LO-CAT® system. In embodiments, AGR comprises a chemical system, such as an amine system.

Product Upgrading Units. System 100 may further comprise product upgrading units. PUs may be any suitable units known in the art for upgrading the Fischer-Tropsch hydrocarbons produced in the Fischer-Tropsch reactors. In embodiments, the PU unit is selected from hydrotreating units, hydrocracking units, fractionators, separators, and combinations thereof.

Process for Fischer-Tropsch Conversion of Synthesis Gas. Description of a process for Fischer-Tropsch conversion of synthesis gas will now be made with reference to FIG. 1.

Multiple Gasifiers. In an embodiment, the invention involves the use of a number of gasifiers at least one greater than a base number of gasifiers required to produce the amount of synthesis gas for full Fischer-Tropsch plant capacity when each of the base number of gasifiers is operated at full capacity. In embodiments, a single additional gasifier is employed. In embodiments, two additional (or ‘spare’) gasifiers are utilized. In the embodiment of FIG. 1, system 100 comprises 5 gasifiers.

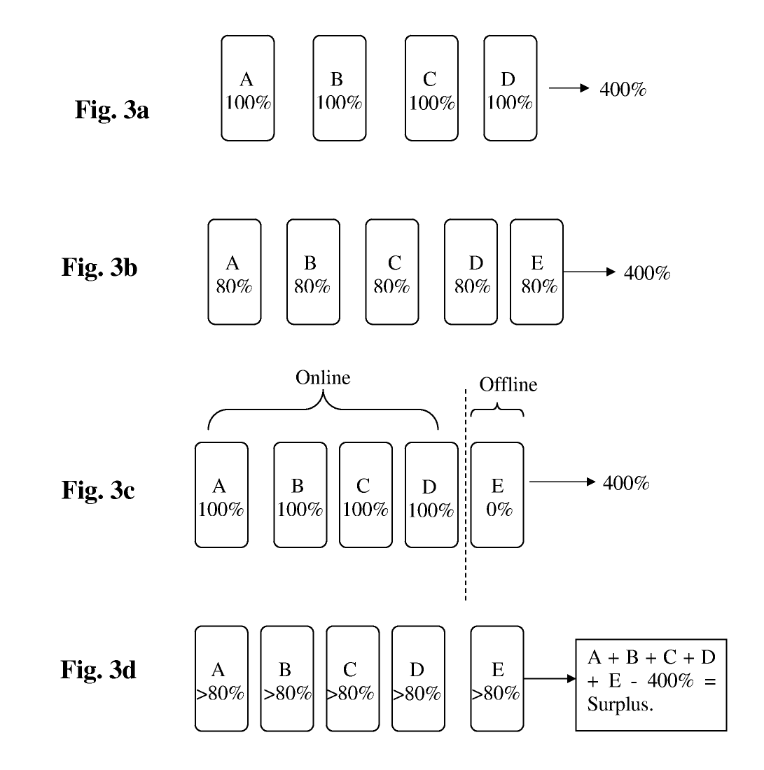

FIG. 3a is a schematic of a series of 4 gasifiers, A, B, C, and D. Assume each gasifier works at 100% capacity, yielding 400% total. Assume also that 400% is the required synthesis gas production for the Fischer-Tropsch plant. According to an embodiment of this disclosure, the system comprises at least one additional gasifier above the base number required to provide 100% of the synthesis gas required for the plant to operate at full capacity when each gasifier is operated at full capacity. As shown in FIG. 3b, according to an embodiment, the series of gasifiers now comprises gasifier E. If, during normal operation, each gasifier A-E works at 80% production, full plant capacity of 400% synthesis gas production may be obtained.

Production of a steady supply of synthesis gas, even in the event that a gasifier must be pulled offline for service/repair/replacement, is permitted by the utilization of at least one additional (‘spare’) gasifier. That is, at least one of gasifiers A-E may be taken offline and the production of synthesis gas from the remaining gasifiers ramped up above the operating capacity used when all the gasifiers are online to maintain the required synthesis gas for the Fischer-Tropsch plant. For example, as shown in FIG. 3c, a gasifier (shown exemplarily as gasifier E) has been taken offline for repair, etc. In this case, the remaining online gasifiers, (gasifiers A-D in FIG. 3c) may be ramped up to 100% capacity each, so that the required plant capacity of 400% synthesis gas is maintained even during this time of gasifier repair. Each of the remaining gasifiers may be ramped up to an operating capacity from just above the normal operating capacity to 100% gasifier capacity. The online gasifiers need not each operate at the same capacity. In this manner, at least one of gasifiers A-E may be taken offline and the remaining gasifiers ramped up to provide the synthesis gas required for full production of hydrocarbons via the Fischer-Tropsch process. In some instances, at least two gasifiers may be taken offline.

It may be desirable to operate the plurality of gasifiers such that excess synthesis gas is produced (e.g., an amount of synthesis gas greater than the amount required for full plant production of Fischer-Tropsch hydrocarbons). For example, as shown in FIG. 3d, gasifiers A-E may be operated at capacities greater than the 80% required for full plant capacity, leading to a surplus synthesis gas production equal to the combined production of gasifiers A, B, C, D, and E minus 400%. This surplus synthesis gas may be stored for later use, etc.

It is to be noted that, although the examples provided here portray each online gasifier operating at the same capacity, it should be readily apparent to one of skill in the art that one or more of the online gasifiers may operate at a different operating capacity.

Thus, in embodiments, one or more gasifier (including the ‘spare’) may be operated at less than the maximum capacity of the gasifier while maintaining 100% plant capacity, or, in other embodiments, the gasifiers may be operated such that greater than 100% of the required synthesis gas is formed. The normal use of an additional gasifier and the optional production of surplus synthesis gas and storage thereof may permit repair/service of gasifiers without reducing plant production and/or may permit storage of synthesis gas for later use.

With synthesis gas, it may be useful to make extra synthesis gas and store it, although the capital expense will be increased due to the use of larger gasifiers. For instance, rather than using 4 gasifiers operating at 100%, the present invention proposes, in normal operation, utilizing 5 gasifiers (i.e., four gasifiers and a ‘spare’ or additional gasifier) operating at a normal capacity of 80% to attain the required throughout, for example. The use of larger gasifiers will incur an increased capital expense, but will allow continuous production should a gasifier need repair or become inoperable. That is, should the need arise, one of the gasifiers may be placed offline for repair or etc, and the remaining online gasifiers operated at higher (e.g., at 100%) capacity to maintain the production of the plant during repair of the offline gasifier.

Additionally, should it be economically feasible to do so, during normal operation, the plurality of the gasifiers may be operated at 100%, and the surplus synthesis gas produced sent for storage, for example, in a storage tank or a salt dome. For example, there are bedrock encased sandstone formations throughout the world in use for natural gas storage. During the summer, when demand is low, excess natural gas is collected and compressed into the porous sandstone with diesel compressors. The sandstone is encased in bedrock so the natural gas does not leak out. When it is cold, and demand is high, the gas can be recovered. In a similar manner, by this invention, surplus synthesis gas created by the normal operation of an additional gasifier may be stored for future use in, for example, a salt dome, should this prove economically desirable.

The salt dome may be a natural formation or a formation created by mining with brine. Steam may be injected into a salt dome, and the salt dissolved and extracted as a liquid. This creates void space within the salt dome. Surplus synthesis gas may be compressed and pumped into such a salt dome for future use.

In some applications, one or more tanks may be used for storage of surplus synthesis gas; however it may cost a substantial amount to build a tank suitable for holding a few plant operation hours worth of synthesis gas. Instead of this, in embodiments of this invention, use of a natural formation to store the synthesis gas is envisioned where applicable (i.e., where the salt dome is adequately located such that the costs associated with transportation, compressing, pumping into the formation, and recovering the gas therefrom, are not economically prohibitive).

In embodiments, a surplus amount of synthesis gas is stored for the event that an unscheduled outage occurs during which a shortage of synthesis gas will be produced. In this manner, the plant may be kept running even should all gasifiers be offline for a period of time.

Multiple Gasifiers and Multiple Fischer-Tropsch Reactors. In an embodiment, the process comprises the use of multiple gasifiers manifolded to multiple Fischer-Tropsch reactors for the steady production of gas throughout a XTL (“anything to liquids”, e.g., CTL or ‘coal to liquids’) plant. In such embodiments, the system comprises at least one more gasifier than the base number required to provide 100% of the amount of synthesis gas needed by the Fischer-Tropsch plant for operation at full capacity when each base gasifier is operated at full production capacity; and at least one more Fischer-Tropsch reactor than the base number of Fischer-Tropsch reactors required to process the amount of synthesis gas processed by the plant at full capacity. The manifolding of processing units together may permit operation of an XTL plant at a steady flow of synthesis gas.

According to this embodiment, multiple gasifiers are used to produce synthesis gas, and the multiple gasifiers are manifolded together and fed to each of a plurality of Fischer-Tropsch reactors. The use of multiple gasifiers and Fischer-Tropsch reactors manifolded together enables turning the plant down or turning the plant up to match plant requirements. In the embodiment of FIG. 1, five gasifiers, gasifiers 1, 2, 3, 4, and 5 are manifolded together, and the synthesis gas produced therein used to feed six Fischer-Tropsch reactors, Fischer-Tropsch reactors 30, 31, 32, 33, 34, and 35. Within gasifiers 1-5, carbonaceous feed is converted into gas comprising synthesis gas. Synthesis gas produced in the gasifiers and introduced into line 10 may be fed into one or more acid gas removal units. In the embodiment of FIG. 1, synthesis gas in line 10 is introduced via lines 6 and 7 into two AGR units, AGRs 20 and 21. Within the acid gas removal unit(s), hydrogen sulfide in the sour gas produced in the gasifiers may be removed. Sweet synthesis gas exiting the AGR(s) may be introduced into the Fischer-Tropsch reactors. In the embodiment of FIG. 1, sweet synthesis gas exiting AGRs 20 and 21 via lines 8 and 9 is introduced via line 22 and valves 11, 12, 13, 14, 15, and 16 into Fischer-Tropsch reactors 30, 31, 32, 33, 34, and 35 respectively.

Within the Fischer-Tropsch reactors, synthesis gas is converted into higher hydrocarbons in the presence of suitable Fischer-Tropsch catalyst. Fischer-Tropsch tailgas may be separated from the Fischer-Tropsch product and recycled to the Fischer-Tropsch reactors (not shown in FIG. 1). Fischer-Tropsch tailgas in product line 50 may be recycled to the Fischer-Tropsch reactors via one or more compressors. In embodiments, utilization of a number of compressors less than the number of Fischer-Tropsch reactors is permitted by this method. In the embodiment of FIG. 1, two recycle compressors 40 and 42 serve to recycle Fischer-Tropsch tailgas from line 50 via lines 51 and 52, valves 17 and 18, and lines 41 and 43 respectively. Thus, recycle compressors 40 and 42 may be used to recycle Fischer-Tropsch tailgas back to the Fischer-Tropsch reactors. By using multiple Fischer-Tropsch reactors, and manifolding them together as in FIGS. 1 and 2, fewer compressors may be permitted, e.g., rather than 1 recycle compressor for each Fischer-Tropsch reactor, recycle compressors 40 and 42 of FIG. 1 and compressors 240 and 242 of FIG. 2 may serve to recycle Fischer-Tropsch tailgas for all 6+Fischer-Tropsch reactors. Two trains of compression may serve all the Fischer-Tropsch reactors.

Even should a Fischer-Tropsch reactor need to be pulled offline, the rates of the remaining reactors can be increased to maintain plant production at full capacity.

In embodiments, the Fischer-Tropsch reactors are isolated so that, in the case of fluidized bed reactors, the bed may be kept fluidized even when pulled offline. The introduction of a fluidizing gas into the offline reactor will prevent/minimize solidification of the bed when synthesis gas flow is stopped and the reactor pulled offline. In embodiments, the fluidizing gas may comprise nitrogen. In other embodiments, the fluidizing gas is synthesis gas.

In embodiments, if a Fischer-Tropsch reactor is shutdown, by manifolding the Fischer-Tropsch reactors together, we can increase rates on the balance of the reactors and minimize if not eliminate the affect on plant production. The number of compressors depends on how much turndown is required.

Fischer-Tropsch product in line 50 may be sent to product upgrading, storage, or other use. Fischer-Tropsch product in line 50 may be introduced into one or more product upgrading unit(s). In the embodiment of FIG. 1, Fischer-Tropsch product is introduced into PU unit 60 via line 53. In the embodiment of FIG. 2, Fischer-Tropsch product is introduced into two product upgrading units, PD 260 and PU 261, via lines 253 and 255 respectively. The PUs serve to upgrade the Fischer-Tropsch product as desired, for example to stabilize the products, and/or to separate Fischer-Tropsch naphtha and/or Fischer-Tropsch diesel from the product in line 50. Upgraded product in line 65 or 265 may be sent for sale. Fischer-Tropsch product may be stored, for example via line 54 in FIG. 1 (or line 254 in FIG. 2), for upgrading at a later time, should there be a single PU that needs to be offline for a time. In this manner, the Fischer-Tropsch process can continue running while a PU is offline.

As per FIG. 1, there may be a single product upgrading unit, and tankage may be used to manage the different rates if the product upgrading unit cannot turn down to a desired point. Alternatively, as shown in FIG. 2, two PU units may be used, whereby, rather than storing product in times when one PU must be taken offline, the remaining online PU can be operated at a higher rate and plant production maintained at 100% product production.

Features/Advantages. Processing units go on-stream and off-stream on a periodic basis. Gasifiers, for example, are only about 82% reliable. It is desirable to design Fischer-Tropsch plants with greater on-stream efficiency and this may be achieved by utilizing multiple units. For example, in FIG. 1, five gasifiers are manifolded together and fed to two AGR units. From the AGR units, the gas flows into six Fischer-Tropsch reactors. After the Fischer-Tropsch reactors, there may be one or more product upgrading (PU) units. In contrast, conventional systems comprising, say, only one AGR and two gasifiers impose special design considerations on the gasifier to maintain production while switching gasifiers. In such instances, it is necessary to be able to simultaneously shut one gasifier down while the other gasifier is brought online to avoid shutting the plant down for any period of time. According to this disclosure, rather than providing multiple independent synthesis gas generation trains, the units are manifolded together with subsequent units (e.g. AGRs) and these subsequent units may be manifolded again for introduction into Fischer-Tropsch reactors. This manifolding may increase plant reliability by enabling operation of various units at various times, and various rates. For example, as discussed above, if equivalent rates are desired, 5 units may be operated at 80% to get the 400% (80% per unit times 5 units) that would be produced by operating 4 units at 100% (100% per unit times 4 units=400%). By manifolding the gas lines together, a steady flow may be maintained during repairs and unexpected outages. Conventional configurations typically result in a production halt/reduction during unexpected outages and/or create complicated operation. The use of multiple gasifiers, AGRs and Fischer-Tropsch reactors manifolded together may permit steady flow of gas to the balance of the plant during outages or repairs.

The system and method of this disclosure permits facilitated startup and turndown by altering the number of online units and/or adjusting the production from each unit. Although additional gasifiers, AGRs, Fischer-Tropsch reactors, and/or PU units are utilized in the disclosed system and method, increasing capital expenditure, the flexibility provided by the additional units during startup, turndown, and equipment repair/maintenance may make up for the increased capital expense.

As discussed hereinabove, in alternative embodiments, various numbers of gasifiers, AGRs, Fischer-Tropsch reactors, and/or PU units are manifolded together according to this invention. The invention is not limited to the number or combination of specific units shown in the attached Figures and specifically discussed herein.

### EXAMPLES

### Example 1

As an example of the benefits of the disclosed system and process, consider the system as shown in FIG. 1. During normal operation, gasifiers 1-5 may be run at 80%. Because the gasifiers are manifolded together, if one gasifier must be taken offline, the remaining online gasifiers may be ramped up to maintain plant production. If the turndown capacity of the AGR is greater than the gasifier, it can operate, albeit marginally, at a minimum rate of 25% of the design synthesis gas capacity (or 12.5% as shown in FIG. 1 with two AGR units). With 5 gasifiers, when the first one is brought on line at 60% of its individual capacity, it will produce 12% of the design synthesis gas allowing a single AGR to be brought online. Similarly with 6+Fischer-Tropsch reactors, operation at 12% of design synthesis gas may be adequate to feed a single Fischer-Tropsch reactor and the plant can be started up easily with minimal flaring or non-beneficial use of synthesis gas. If the PU unit has a turndown capacity of say 20% of the synthesis gas, the 8% difference may be made up by feeding from tankage rather than sparing the PU unit. Subsequent gasifiers may then be brought online. Once multiple gasifiers are operating, if something happens to a gasifier and it is shutdown, the process can remain online. Product may be stored as shown for upgrading at a later time, should there be a single PU that needs to be offline for a time, without shutting down the process.

While preferred embodiments of the invention have been shown and described, modifications thereof can be made by one skilled in the art without departing from the spirit and teachings of the invention. The embodiments described herein are exemplary only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, and so forth). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The disclosures of all patents, patent applications, and publications cited herein are hereby incorporated by reference, to the extent they provide exemplary, procedural or other details supplementary to those set forth herein.

What is claimed is:

1. A method of producing synthesis gas via gasification in a Fischer-Tropsch plant, the method comprising:
providing a number of gasifiers, the number of gasifiers provided being at least one more than the base number required to provide 100% plant capacity of synthesis gas when each gasifier is operated at 100% gasifier capacity.

2. The method of claim 1 further comprising normally operating all of the number of gasifiers at a capacity whereby greater than 100% of the synthesis gas required by the plant is produced.

3. The method of claim 2 further comprising storing the surplus synthesis gas in a tank.

4. The method of claim 2 further comprising storing the surplus synthesis gas in a salt dome.

5. The method of claim 1 further comprising normally operating each of the number of gasifiers at less than 100% gasifier capacity while maintaining 100% Fischer-Tropsch plant capacity.

6. A method of continually producing synthesis gas via gasification of a carbonaceous feed in a Fischer-Tropsch plant, the method comprising:
providing a number of gasifiers, the number of gasifiers provided being at least one more than the base number required to provide 100% plant capacity of synthesis gas when each gasifier is operated at 100% gasifier capacity; and
adjusting the amount of synthesis gas produced by adjusting the number of online gasifiers, the flow rate of carbonaceous feed to each gasifier, or a combination thereof.

7. The method of claim 6 wherein adjusting the amount of synthesis gas produced further comprises taking at least one gasifier offline and increasing the amount of synthesis gas produced by the remaining online gasifiers such that 100% Fischer-Tropsch plant capacity is maintained.

8. A method of maintaining full production of product in a Fischer-Tropsch process, the method comprising:
manifolding a plurality of Fischer-Tropsch reactors to a plurality of gasifiers in a Fischer-Tropsch process having a required synthesis gas production flow rate for full capacity, whereby the plurality of gasifiers minus one is capable of producing the required synthesis gas flow rate, and wherein the plurality of Fischer-Tropsch reactors minus one is capable of producing full capacity of product.

9. The method of claim 8 wherein the turndown capacity of each of the plurality of Fischer-Tropsch reactors is such that a single Fischer-Tropsch reactor is capable of being operated with an amount of synthesis gas produced by one of the plurality of gasifiers during startup.

10. The method of claim 8 further comprising recycling at least a portion of a Fischer-Tropsch tail gas produced in the Fischer-Tropsch reactors to at least one of the Fischer-Tropsch reactors via at least one recycle compressor.

11. The method of claim 10 wherein the number of recycle compressors is less than the number of Fischer-Tropsch reactors.

12. The method of claim 8 wherein, during startup, a single gasifier and a single Fischer-Tropsch reactor may be brought online prior to the remaining Fischer-Tropsch reactors and the remaining gasifiers being brought online.

13. The method of claim 8 further comprising:
introducing the synthesis gas produced in the gasifiers to at least one acid gas removal unit upstream of the plurality of Fischer-Tropsch reactors; and
introducing at least a portion of the Fischer-Tropsch product from the plurality of Fischer-Tropsch reactors into at least one product upgrading unit.

14. The method of claim 13 wherein, during startup, a single gasifier, a single acid gas removal unit, a single Fischer-Tropsch reactor, and a single product upgrading unit may be brought online prior to the remaining units being brought online.

15. The method of claim 13 wherein introducing the synthesis gas produced in the gasifiers to at least one acid gas removal unit comprises introducing the synthesis gas produced in the gasifiers into a plurality of acid gas removal units, wherein at least one Fischer-Tropsch reactor, at least one gasifier, and at least one acid gas removal unit may be taken offline while maintaining full plant capacity.

16. The method of claim 13 further comprising introducing at least a portion of the Fischer-Tropsch product into a storage unit if the amount of Fischer-Tropsch product produced in the plurality of Fischer-Tropsch reactors is greater than the capacity of the online product upgrading units.

17. The method of claim 8 comprising utilization of at least 6 Fischer-Tropsch reactors and at least 5 gasifiers.

18. The method of claim 17 further comprising utilization of at least two product upgrading units.

19. The method of claim 18 further comprising utilization of at least two acid gas removal units.

20. A Fischer-Tropsch system comprising:
a plurality of gasifiers manifolded together, wherein the plurality of gasifiers have a synthesis gas production capacity greater than the required synthesis gas for operation of the Fischer-Tropsch system at full capacity, whereby at least one of the plurality of gasifiers may be taken offline and the remaining gasifiers ramped up to maintain full plant production.

21. The system of claim 20 further comprising at least one of acid gas removal unit downstream of the plurality of gasifiers for the removal of sulfur.

22. The system of claim 21 comprising a plurality of acid gas removal units.

23. The Fischer-Tropsch system of claim 20 further comprising a plurality of Fischer-Tropsch reactors manifolded to the plurality of gasifiers, whereby at least one of the plurality of reactors may be taken offline and the remaining reactors ramped up to maintain full plant production.

24. The system of claim 23 further comprising a number of recycle compressors adapted to return Fischer-Tropsch tail gas to at least one of the Fischer-Tropsch reactors, wherein the number of recycle compressors is at least one less than the number of Fischer-Tropsch reactors.

25. The system of claim 24 wherein the system comprises at least 4 Fischer-Tropsch reactors and at least two recycle compressors.

26. The system of claim 23 further comprising at least one product upgrading unit downstream of the plurality of Fischer-Tropsch reactors.

27. The system of claim 26 further comprising storage for Fischer-Tropsch product should at least one product upgrading unit be taken offline.

28. The system of claim 26 comprising a single product upgrading unit and storage for Fischer-Tropsch product should the single product upgrading unit be taken offline

29. The system of claim 26 further comprising at least 2 acid gas removal units and comprising at least 5 gasifiers, at least 4 Fischer-Tropsch reactors, and at least one product upgrading unit.
